(19) United States
(12) Patent Application Publication
(10) Pub. No.: US 2025/0283109 A1
(43) Pub. Date: Sep. 11, 2025

(54) PLASMIDS AND METHODS OF PRODUCTION OF ADENO-ASSOCIATED VIRUSES

(71) Applicant: CAPSIDA, INC., Thousand Oaks, CA (US)

(72) Inventors: Nicholas S. Goeden, Thousand Oaks, CA (US); Laura Adamson, Thousand Oaks, CA (US)

(21) Appl. No.: 18/284,404

(22) PCT Filed: Apr. 13, 2022
(86) PCT No.: PCT/US2022/024600

§ 371 Date: Sep. 27, 2023

Related U.S. Application Data
(60) Provisional application No. 63/174,731, filed on Apr. 14, 2021.

(51) Int. Cl. C12N 15/86

(52) U.S. Cl. CPC C12N 15/86; C12N 2750/14143; C12N 2750/14151; C12N 2830/003

(57) ABSTRACT

Compositions and methods of the invention provide plasmids and methods for efficient production of recombinant adeno-associated viruses (rAAVs) for use in numerous areas including research and therapeutic applications of gene transduction. Plasmids of the invention can include rep-cap plasmids comprising rep and cap genes or portions thereof derived from any AAV serotype or engineered capsid. In certain embodiments, rep and/or cap genes may be derived from AAV2, AAV9, and AAV5 serotypes.

### BACKGROUND

Recombinant adeno-associated viruses (rAAVs) are widely used as vectors for gene delivery in therapeutic applications because of their ability to transduce both dividing and non-dividing cells, their long-term persistence as episomal DNA in infected cells, and their low immunogenicity. These characteristics make them appealing for applications in therapeutic applications, such as gene therapy. However, methods for production or manufacturing of such rAAVs for experimental and clinical use are not well developed.

### SUMMARY

The application contains a sequence listing which has been submitted in text format and is hereby incorporated by reference in its entirety. The sequence listing file was created on CAPS-011-01US-SEQ-ST25.txt was created on Dec. 1, 2024 and is 26 KB in size.

Compositions and methods of the invention provide plasmids and methods for efficient production of rAAVs for use in numerous areas including research and therapeutic applications of gene transduction. Plasmids of the invention can include rep-cap plasmids comprising rep and cap genes or portions thereof derived from any AAV serotype or engineered capsid. In certain embodiments, rep and/or cap genes may be derived from AAV2, AAV9, and AAV5 serotypes. Plasmids may include a tetracycline-inducible expression system comprising a tetracycline-controlled transactivator (tTA)-Tetracycline response element (TRE)-based inducible amplification loop to increase virus production. In various embodiments, the TRE may be tet6 or tet8 derived. Rep-cap plasmids of the invention may include helper genes or helper genes may be provided in a separate helper plasmid. A third plasmid may include a gene of interest flanked by inverted terminal repeats. Rep-cap plasmids of the invention may be introduced into a cell along with one or two additional plasmids (depending on whether helper genes are included in the rep-cap plasmid or a separate helper plasmid) in order to produce a rAAV including the gene of interest. Such produced rAAVs can then be used for gene therapy to introduce the gene of interest into cells for research or medical purposes. In certain embodiments, the gene of interest may encode a protein associated with a particular disease such that translation of the rAAV encoded protein may be used to treat the disease.

Aspects of the invention include adeno-associated virus (AAV) rep-cap plasmids comprising an AAV2 rep gene or portions thereof, a cap gene, a tetracycline-inducible expression system in between the AAV2 rep gene and the cap gene, and an AAV5 p41 promoter. The AAV rep-cap plasmid can comprise SEQ ID NO: 1 or SEQ ID NO: 2. The plasmid may further comprise one or more helper genes. In certain embodiments, the one or more helper genes may include E4, E2a, or VA.

In certain aspects, methods of the invention may include manufacturing an adeno-associated virus (AAV). Steps of the method may include introducing an AAV rep-cap plasmid comprising SEQ ID NO: 1 or SEQ ID NO: 2 into a cell. Methods may further include introducing a helper plasmid comprising one or more helper genes into the cell. In certain embodiments, the AAV rep-cap plasmid may further comprise one or more helper genes. The one or more helper genes may include E4, E2a, or VA.

In certain embodiments, methods may include introducing a plasmid comprising a gene of interest flanked by inverse terminal repeats. The gene of interest may be a transgene. The gene of interest may encode a protein associated with a disease. In various embodiments, the cell may be mammalian, may be immortalized, may be an embryonic stem cell, may be a human embryonic stem cell, or may be a human embryonic kidney 293 (HEK-293) cell.

### DETAILED DESCRIPTION

Plasmids and methods of the invention relate to producing recombinant adeno-associated viruses (rAAVs) in producing cells. RAAVs are widely used as vectors for gene delivery in therapeutic applications because of their ability to transduce both dividing and non-dividing cells, their long-term persistence as episomal DNA in infected cells, and their low immunogenicity. Protocols for producing recombinant AAVs are established and can include the transfection of a cell (e.g., an HEK-293 cell) with two or three plasmids. An AAV rep-cap plasmid and a plasmid including the gene of interest or library flanked by inverted terminal repeats are required using such methods. In order for the cell to produce the desired AAVs including the gene of interest, certain helper genes from adenovirus are required and can be introduced in a separate helper plasmid or included in the rep-cap plasmid in certain protocols. Modified iCAP rep-cap plasmids and methods for transfecting producing cells and producing AAVs therewith are known and described, for example, in Challis, et al., 2019, Systemic AAV vectors for widespread and targeted gene delivery in rodents, Nat Protoc, 14:379-414 and Deverman, et al., 2016, Cre-dependent selection yields AAV variants for widespread gene transfer to the adult brain, Nat Biotechnol, 34 (2): 204-209; the content of each of which is incorporated herein by reference.

A modified rep-cap plasmid (e.g, pUCmini-iCAP-PHP), a helper plasmid (e.g., pHelper), and a plasmid containing the gene of interest (e.g., pAAV) are transfected into producing cells (e.g., HEK293T cells). As described in Challis, modified iCAP plasmids may include a tetracycline-controlled transactivator (tTA)-Tetracycline response element (TRE)-based inducible amplification loop to increase virus production.

Rep-cap plasmids of the invention may be modified by substituting or modifying an existing capsid gene to encode a targeting protein providing preferential targeting for a specific cell or tissue. When introduced together into producer cells, this plasmid can be used to package an rAAV genome into the targeting protein and/or capsid. Producer cells can be any cell type possessing the genes necessary to promote AAV genome replication, capsid assembly and packaging. Preferred producer cells are HEK-293 cells, or derivatives, HELA cells or insect cells together with helper virus or a second plasmid encoding the helper virus genes known to promote rAAV genome replication. In some embodiments, an AAV rep-cap helper sequence can be modified to introduce a tetracycline-inducible expression system in between the rep and the cap gene to increase capsid expression and virus production. In some embodiments, a tetracycline transactivator cDNA, poly adenylation sequence, tetracycline responsive element and AAV5 p41 promoter and AAV2 splicing regulatory elements contained within the AAV2 rep gene are inserted between the rep gene and the gene encoding the capsid or targeting protein. Use of an inducible rep-cap plasmid when making rAAV has been shown to provide 1.5-2-fold more virus than a non-inducable AAV2/9 rep-cap plasmid.

rAAVs produced using plasmids and methods of the invention can be used to introduce a variety of genetic material into cells. In certain embodiments, the transduced gene of interest may encode a protein associated with a disease. Examples of treating such diseases using engineered rAAVs are disclosed in, for example, U.S. App Ser. Nos. 63/068,606, 63/068,620, 63/068,627, 63/068,636, and Ser. No. 16/582,635, the contents of each of which is incorporated herein by reference.

Rep-cap plasmids of the invention can include any of a variety of rep and cap sequences or portions thereof from any AAV serotypes or engineered capsids (e.g., AAV2, AAV5, and AAV9). Exemplary AAV2 Rep sequence portions are provided in SEQ ID NOS: 3, 4, 9, and 11. As noted, plasmids may include a tetracycline-inducible expression system comprising a tetracycline-controlled transactivator (tTA)-Tetracycline response element (TRE)-based inducible amplification loop to increase virus production. An exemplary tTA is provided in SEQ ID NO: 5. Exemplary TRE sequences are provided in SEQ ID NO: 7 (tet8) and SEQ ID NO: 12 (tet6). An exemplary hABGH sequence is provided in SEQ ID NO: 6 and may be included in plasmids of the invention. Plasmids can include an AAV5 rep portion or p41 promoter as exemplified in SEQ ID NO: 8. An exemplary cap sequence is provided in SEQ ID NO: 10.

Exemplary rep-cap plasmid sequences including the aforementioned components are provided in SEQ ID NO: 1 (having a tet8 TRE) and SEQ ID NO: 2 (having a TRE).

AAV9-ICAP tet8 TRE-
SEQ ID NO: 1
ATGCCGGGGTTTTACGAGATTGTGATTAAGGTCCCCAGCGACCTT
GACGAGCATCTGCCCGGCATTTCTGACAGCTTTGTGAACTGGGTG
GCCGAGAAGGAATGGGAGTTGCCGCCAGATTCTGACATGGATCTG
AATCTGATTGAGCAGGCACCCCTGACCGTGGCCGAGAAGCTGCAG
CGCGACTTTCTGACGGAATGGCGCCGTGTGAGTAAGGCCCCGGAG
GCTCTTTTCTTTGTGCAATTTGAGAAGGGAGAGAGCTACTTCCAC
ATGCACGTGCTCGTGGAAACCACCGGGGTGAAATCCATGGTTTTG
GGACGTTTCCTGAGTCAGATTCGCGAAAAACTGATTCAGAGAATT
TACCGCGGGATCGAGCCGACTTTGCCAAACTGGTTCGCGGTCACA
AAGACCAGAAATGGCGCCGGAGGCGGGAACAAGGTGGTGGATGAG
TGCTACATCCCCAATTACTTGCTCCCCAAAACCCAGCCTGAGCTC
CAGTGGGCGTGGACTAATATGGAACAGTATTTAAGCGCCTGTTTG
AATCTCACGGAGCGTAAACGGTTGGTGGCGCAGCATCTGACGCAC
GTGTCGCAGACGCAGGAGCAGAACAAAGAGAATCAGAATCCCAAT
TCTGATGCGCCGGTGATCAGATCAAAAACTTCAGCCAGGTACATG
GAGCTGGTCGGGTGGCTCGTGGACAAGGGGATTACCTCGGAGAAG
CAGTGGATCCAGGAGGACCAGGCCTCATACATCTCCTTCAATGCG
GCCTCCAACTCGCGGTCCCAAATCAAGGCTGCCTTGGACAATGCG
GGAAAGATTATGAGCCTGACTAAAACCGCCCCCGACTACCTGGTG
GGCCAGCAGCCCGTGGAGGACATTTCCAGCAATCGGATTTATAAA
ATTTTGGAACTAAACGGGTACGATCCCCAATATGCGGCTTCCGTC
TTTCTGGGATGGGCCACGAAAAAGTTCGGCAAGAGGAACACCATC
TGGCTGTTTGGGCCTGCAACTACCGGGAAGACCAACATCGCGGAG
GCCATAGCCCACACTGTGCCCTTCTACGGGTGCGTAAACTGGACC
AATGAGAACTTTCCCTTCAACGACTGTGTGGACAAGATGGTGATC
TGGTGGGAGGAGGGGAAGATGACCGCCAAGGTCGTGGAGTCGGCC
AAAGCCATTCTCGGAGGAAGCAAGGTGCGCGTGGACCAGAAATGC
AAGTCCTCGGCCCAGATAGACCCGACTCCCGTGATCGTCACCTCC
AACACCAATATGTGCGCCGTGATTGACGGGAACTCAACGACCTTC
GAACACCAGCAGCCGTTGCAAGACCGGATGTTCAAATTTGAACTC
ACCCGCCGTCTGGATCATGACTTTGGGAAGGTCACCAAGCAGGAA
GTCAAAGACTTTTTCCGGTGGGCAAAGGATCACGTGGTTGAGGTG
GAGCATGAATTCTACGTCAAAAAGGGTGGAGCCAAGAAAAGACCC
GCCCCCAGTGACGCAGATATAAGTGAGCCCAAACGGGTGCGCGAG
TCAGTTGCGCAGCCATCGACGTCAGACGCGGAAGCTTCGATCAAC
TACGCGGACAGGTACCAAAACAAATGTTCTCGTCACGTGGGCATG
AATCTGATGCTGTTTCCCTGCAGACAATGCGAGAGACTGAATCAG
AATTCAAATATCTGCTTCACTCACGGTGTCAAAGACTGTTTAGAG
TGCTTTCCCGTGTCAGAATCTCAACCCGTTTCTGTCGTCAAAAAG
GCGTATCAGAAACTGTGCTACATTCATCACATCATGGGAAAGGTG
CCAGACGCTTGCACTGCTTGCGACCTGGTCAATGTGGACTTGGAT
GACTGTGTTTCTGAACAATAAATGACTTAAACCAGGTATGAGTCG
GCTGGATAAATCTAAAGTCATAAACGGCGCTCTGGAATTACTCAA
TGAAGTCGGTATCGAAGGCCTGACGACAAGGAAACTCGCTCAAAA
GCTGGGAGTTGAGCAGCCTACCCTGTACTGGCACGTGAAGAACAA
GCGGGCCCTGCTCGATGCCCTGGCCATCGAGATGCTGGACAGGCA
TCATACCCACTTCTGCCCCCTGGAAGGCGAGTCATGGCAAGACTT
TCTGCGGAACAACGCCAAGTCATTCCGCTGTGCTCTCCTCTCACA
TCGCGACGGGGCTAAAGTGCATCTCGGCACCCGCCCAACAGAGAA
ACAGTACGAAACCCTGGAAAATCAGCTCGCGTTCCTGTGTCAGCA
AGGCTTCTCCCTGGAGAACGCACTGTACGCTCTGTCCGCCGTGGG
CCACTTTACACTGGGCTGCGTATTGGAGGAACAGGAGCATCAAGT
AGCAAAAGAGGAAAGAGAGACACCTACCACCGATTCTATGCCCCC
ACTTCTGAGACAAGCAATTGAGCTGTTCGACCGGCAGGGAGCCGA
ACCTGCCTTCCTTTTCGGCCTGGAACTAATCATATGTGGCCTGGA
GAAACAGCTAAAGTGCGAAAGCGGCGGGCCGGCCGACGCCCTTGA
CGATTTTGACTTAGACATGCTCCCAGCCGATGCCCTTGACGACTT
TGACCTTGATATGCTGCCTGCTGACGCTCTTGACGATTTTGACCT
TGACATGCTCCCCGGGTAAATGCATGAATTCGATCTAGAGGGCCC
TATTCTATAGTGTCACCTAAATGCTAGAGCTCGCTGATCAGCCTC
GACTGTGCCTTCTAGTTGCCAGCCATCTGTTGTTTGCCCCTCCCC
CGTGCCTTCCTTGACCCTGGAAGGTGCCACTCCCACTGTCCTTTC
CTAATAAAATGAGGAAATTGCATCGCATTGTCTGAGTAGGTGTCA
TTCTATTCTGGGGGGTGGGGTGGGGCAGGACAGCAAGGGGGAGGA
TTGGGAAGACAATAGCAGGCATGCTGGGGATGCGGTGGGCTCTAT
GGCTTCTGAGGCGGAAAGAACCAGCTGGGGCTCGAATCAAGCTAT
CAAGTGCCACCTGACGTCTCCCTATCAGTGATAGAGAAGTCGACA
CGTCTCGAGCTCCCTATCAGTGATAGAGAAGGTACGTCTAGAACG
TCTCCCTATCAGTGATAGAGAAGTCGACACGTCTCGAGCTCCCTA
TCAGTGATAGAGAAGGTACGTCTAGAACGTCTCCCTATCAGTGAT
AGAGAAGTCGACACGTCTCGAGCTCCCTATCAGTGATAGAGAAGG
TACGTCTAGAACGTCTCCCTATCAGTGATAGAGAAGTCGACACGT
CTCGAGCTCCCTATCAGTGATAGAGAAGGTACCCCCTATATAAGC
AGAGAGATCTGTTCAAATTTGAACTGACTAAGCGGCTCCCGCCAG
ATTTTGGCAAGATTACTAAGCAGGAAGTCAAGGACTTTTTTGCTT
GGGCAAAGGTCAATCAGGTGCCGGTGACTCACGAGTTTAAAGTTC
CCAGGGAATTGGCGGGAACTAAAGGGGCGGAGAAATCTCTAAAAC
GCCCACTGGGTGACGTCACCAATACTAGCTATAAAAGTCTGGAGA
AGCGGGCCAGGCTCTCATTTGTTCCCGAGACGCCTCGCAGTTCAG
ACGTGACTGTTGATCCCGCTCCTCTGCGACCGCTAGCTTCGATCA
ACTACGCAGACAGGTACCAAAACAAGTGTTCTCGTCACGTGGGCA
TTAATCTGATTCTGTTTCCCTGCAGACAATGCGAGAGAATGAATC
AGAACTCAAATATCTGCTTCACTCACGGACAGAAAGACTGTTTAG
AGTGCTTTCCCGTGTCAGAATCTCAACCCGTTTCTGTCGTCAAAA
AGGCGTATCAGAAACTGTGCTACATTCATCATATCATGGGAAAGG
TGCCAGACGCTTGCACTGCCTGCGATCTGGTCAATGTGGATTTGG
ATGACTGCATCTTTGAACAATAAATGACTTAAGCCAGGTATGGCT
GCCGATGGTTATCTTCCAGATTGGCTCGAGGACAACCTTAGTGAA
GGAATTCGCGAGTGGTGGGCTTTGAAACCTGGAGCCCCTCAACCC
AAGGCAAATCAACAACATCAAGACAACGCTAGAGGTCTTGTGCTT
CCGGGTTACAAATACCTTGGACCCGGCAACGGACTCGACAAGGGG
GAGCCGGTCAACGCAGCAGACGCGGCGGCCCTCGAGCACGACAAA
GCCTACGACCAGCAGCTCAAGGCCGGAGACAACCCGTACCTCAAG
TACAACCACGCCGACGCCGAGTTCCAGGAGCGGCTCAAAGAAGAT
ACGTCTTTTGGGGGCAACCTCGGGCGAGCAGTCTTCCAGGCCAAA
AAGAGGCTTCTTGAACCTCTTGGTCTGGTTGAGGAAGCGGCTAAG
ACGGCTCCTGGAAAGAAGAGGCCTGTAGAGCAGTCTCCTCAGGAA
CCGGACTCCTCCGCGGGTATTGGCAAATCGGGTGCACAGCCCGCT
AAAAAGAGACTCAATTTCGGTCAGACTGGCGACACAGAGTCAGTC
CCAGACCCTCAACCAATCGGAGAACCTCCCGCAGCCCCCTCAGGT
GTGGGATCTCTTACAATGGCTTCAGGTGGTGGCGCACCAGTGGCA
GACAATAACGAAGGTGCCGATGGAGTGGGTAGTTCCTCGGGAAAT
TGGCATTGCGATTCCCAATGGCTGGGGGACAGAGTCATCACCACC
AGCACCCGAACCTGGGCCCTGCCCACCTACAACAATCACCTCTAC
AAGCAAATCTCCAACAGCACATCTGGAGGATCTTCAAATGACAAC
GCCTACTTCGGCTACAGCACCCCCTGGGGGTATTTTGACTTCAAC
AGATTCCACTGCCACTTCTCACCACGTGACTGGCAGCGACTCATC
AACAACAACTGGGGATTCCGGCCTAAGCGACTCAACTTCAAGCTC
TTTAACATTCAGGTCAAAGAGGTTACGGACAACAATGGAGTCAAG
ACCATCGCCAATAACCTTACCAGCACGGTCCAGGTCTTCACGGAC
TCAGACTATCAGCTCCCGTACGTGCTCGGGTCGGCTCACGAGGGC
TGCCTCCCGCCGTTCCCAGCGGACGTTTTCATGATTCCTCAGTAC
GGGTATCTGACGCTTAATGATGGAAGCCAGGCCGTGGGTCGTTCG
TCCTTTTACTGCCTGGAATATTTCCCGTCGCAAATGCTAAGAACG
GGTAACAACTTCCAGTTCAGCTACGAGTTTGAGAACGTACCTTTC
CATAGCAGCTACGCTCACAGCCAAAGCCTGGACCGACTAATGAAT
CCACTCATCGACCAATACTTGTACTATCTCTCTAGAACTATTAAC
GGTTCTGGACAGAATCAACAAACGCTAAAATTCAGTGTGGCCGGA
CCCAGCAACATGGCTGTCCAGGGAAGAAACTACATACCTGGACCC
AGCTACCGACAACAACGTGTCTCAACCACTGTGACTCAAAACAAC
AACAGCGAATTTGCTTGGCCTGGAGCTTCTTCTTGGGCTCTCAAT
GGACGTAATAGCTTGATGAATCCTGGACCTGCTATGGCCTCTCAC
AAAGAAGGAGAGGACCGTTTCTTTCCTTTGTCTGGATCTTTAATT
TTTGGCAAACAAGGTACTGGCAGAGACAACGTGGATGCGGACAAA
GTCATGATAACCAACGAAGAAGAAATTAAAACTACTAACCCGGTA
GCAACGGAGTCCTATGGACAAGTGGCCACAAACCACCAGAGTGCC
CAAGCACAGGCGCAGACCGGTTGGGTTCAAAACCAAGGAATACTT
CCGGGTATGGTTTGGCAGGACAGAGATGTGTACCTGCAAGGACCC
ATTTGGGCCAAAATTCCTCACACGGACGGCAACTTTCACCCTTCT
CCGCTGATGGGAGGGTTTGGAATGAAGCACCCGCCTCCTCAGATC
CTCATCAAAAACACACCTGTACCTGCGGATCCTCCAACGGCCTTC
AACAAGGACAAGCTGAACTCTTTCATCACCCAGTATTCTACTGGT
CAAGTCAGCGTGGAGATCGAGTGGGAGCTGCAGAAGGAAAACAGC
AAGCGCTGGAACCCGGAGATCCAGTACACTTCCAACTATTACAAG
TCTAATAATGTTGAATTTGCTGTTAATACTGAAGGTGTATATAGT
GAACCCCGCCCCATTGGCACCAGATACCTGACTCGTAATCTGTAA
GTCGACTTGCTTGTTAATCAATAAACCGTTTAATTCGTTTCAGTT
GAACTTTGGTCTCTGCGAAGGGCAATTCGTTTAAACCTGCAGGAC
TAGAGGTCCTGTATTAGAGGTCACGTGAGTGTTTTGCGACATTTT
GCGACACCATGTGGTCACGCTGGGTATTTAAGCCCGAGTGAGCAC
GCAGGGTCTCCATTTTGAAGCGGGAGGTTTGAACGCGCAGCCGCC
AAV9-ICAP tet6 TRE-
SEQ ID NO: 2
ATGCCGGGGTTTTACGAGATTGTGATTAAGGTCCCCAGCGACCTT
GACGAGCATCTGCCCGGCATTTCTGACAGCTTTGTGAACTGGGTG
GCCGAGAAGGAATGGGAGTTGCCGCCAGATTCTGACATGGATCTG
AATCTGATTGAGCAGGCACCCCTGACCGTGGCCGAGAAGCTGCAG
CGCGACTTTCTGACGGAATGGCGCCGTGTGAGTAAGGCCCCGGAG
GCTCTTTTCTTTGTGCAATTTGAGAAGGGAGAGAGCTACTTCCAC
ATGCACGTGCTCGTGGAAACCACCGGGGTGAAATCCATGGTTTTG
GGACGTTTCCTGAGTCAGATTCGCGAAAAACTGATTCAGAGAATT
TACCGCGGGATCGAGCCGACTTTGCCAAACTGGTTCGCGGTCACA
AAGACCAGAAATGGCGCCGGAGGCGGGAACAAGGTGGTGGATGAG
TGCTACATCCCCAATTACTTGCTCCCCAAAACCCAGCCTGAGCTC
CAGTGGGCGTGGACTAATATGGAACAGTATTTAAGCGCCTGTTTG
AATCTCACGGAGCGTAAACGGTTGGTGGCGCAGCATCTGACGCAC
GTGTCGCAGACGCAGGAGCAGAACAAAGAGAATCAGAATCCCAAT
TCTGATGCGCCGGTGATCAGATCAAAAACTTCAGCCAGGTACATG
GAGCTGGTCGGGTGGCTCGTGGACAAGGGGATTACCTCGGAGAAG
CAGTGGATCCAGGAGGACCAGGCCTCATACATCTCCTTCAATGCG
GCCTCCAACTCGCGGTCCCAAATCAAGGCTGCCTTGGACAATGCG
GGAAAGATTATGAGCCTGACTAAAACCGCCCCCGACTACCTGGTG
GGCCAGCAGCCCGTGGAGGACATTTCCAGCAATCGGATTTATAAA
ATTTTGGAACTAAACGGGTACGATCCCCAATATGCGGCTTCCGTC
TTTCTGGGATGGGCCACGAAAAAGTTCGGCAAGAGGAACACCATC
TGGCTGTTTGGGCCTGCAACTACCGGGAAGACCAACATCGCGGAG
GCCATAGCCCACACTGTGCCCTTCTACGGGTGCGTAAACTGGACC
AATGAGAACTTTCCCTTCAACGACTGTGTGGACAAGATGGTGATC
TGGTGGGAGGAGGGGAAGATGACCGCCAAGGTCGTGGAGTCGGCC
AAAGCCATTCTCGGAGGAAGCAAGGTGCGCGTGGACCAGAAATGC
AAGTCCTCGGCCCAGATAGACCCGACTCCCGTGATCGTCACCTCC
AACACCAATATGTGCGCCGTGATTGACGGGAACTCAACGACCTTC
GAACACCAGCAGCCGTTGCAAGACCGGATGTTCAAATTTGAACTC
ACCCGCCGTCTGGATCATGACTTTGGGAAGGTCACCAAGCAGGAA
GTCAAAGACTTTTTCCGGTGGGCAAAGGATCACGTGGTTGAGGTG
GAGCATGAATTCTACGTCAAAAAGGGTGGAGCCAAGAAAAGACCC
GCCCCCAGTGACGCAGATATAAGTGAGCCCAAACGGGTGCGCGAG
TCAGTTGCGCAGCCATCGACGTCAGACGCGGAAGCTTCGATCAAC
TACGCGGACAGGTACCAAAACAAATGTTCTCGTCACGTGGGCATG
AATCTGATGCTGTTTCCCTGCAGACAATGCGAGAGACTGAATCAG
AATTCAAATATCTGCTTCACTCACGGTGTCAAAGACTGTTTAGAG
TGCTTTCCCGTGTCAGAATCTCAACCCGTTTCTGTCGTCAAAAAG
GCGTATCAGAAACTGTGCTACATTCATCACATCATGGGAAAGGTG
CCAGACGCTTGCACTGCTTGCGACCTGGTCAATGTGGACTTGGAT
GACTGTGTTTCTGAACAATAAATGACTTAAACCAGGTATGAGTCG
GCTGGATAAATCTAAAGTCATAAACGGCGCTCTGGAATTACTCAA
TGAAGTCGGTATCGAAGGCCTGACGACAAGGAAACTCGCTCAAAA
GCTGGGAGTTGAGCAGCCTACCCTGTACTGGCACGTGAAGAACAA
GCGGGCCCTGCTCGATGCCCTGGCCATCGAGATGCTGGACAGGCA
TCATACCCACTTCTGCCCCCTGGAAGGCGAGTCATGGCAAGACTT
TCTGCGGAACAACGCCAAGTCATTCCGCTGTGCTCTCCTCTCACA
TCGCGACGGGGCTAAAGTGCATCTCGGCACCCGCCCAACAGAGAA
ACAGTACGAAACCCTGGAAAATCAGCTCGCGTTCCTGTGTCAGCA
AGGCTTCTCCCTGGAGAACGCACTGTACGCTCTGTCCGCCGTGGG
CCACTTTACACTGGGCTGCGTATTGGAGGAACAGGAGCATCAAGT
AGCAAAAGAGGAAAGAGAGACACCTACCACCGATTCTATGCCCCC
ACTTCTGAGACAAGCAATTGAGCTGTTCGACCGGCAGGGAGCCGA
ACCTGCCTTCCTTTTCGGCCTGGAACTAATCATATGTGGCCTGGA
GAAACAGCTAAAGTGCGAAAGCGGCGGGCCGGCCGACGCCCTTGA
CGATTTTGACTTAGACATGCTCCCAGCCGATGCCCTTGACGACTT
TGACCTTGATATGCTGCCTGCTGACGCTCTTGACGATTTTGACCT
TGACATGCTCCCCGGGTAAATGCATGAATTCGATCTAGAGGGCCC
TATTCTATAGTGTCACCTAAATGCTAGAGCTCGCTGATCAGCCTC
GACTGTGCCTTCTAGTTGCCAGCCATCTGTTGTTTGCCCCTCCCC
CGTGCCTTCCTTGACCCTGGAAGGTGCCACTCCCACTGTCCTTTC
CTAATAAAATGAGGAAATTGCATCGCATTGTCTGAGTAGGTGTCA
TTCTATTCTGGGGGGTGGGGTGGGGCAGGACAGCAAGGGGGAGGA
TTGGGAAGACAATAGCAGGCATGCTGGGGATGCGGTGGGCTCTAT
GGCTTCTGAGGCGGAAAGAACCAGCTGGGGCTCGAATCAAGCTAT
CAAGTGCCACCTGACGTCTCCCTATCAGTGATAGAGAAGTCGACA
CGTCTCGAGCTCCCTATCAGTGATAGAGAAGGTACGTCTAGAACG
TCTCCCTATCAGTGATAGAGAAGTCGACACGTCTCGAGCTCCCTA
TCAGTGATAGAGAAGGTACGTCTAGAACGTCTCCCTATCAGTGAT
AGAGAAGTCGACACGTCTCGAGCTCCCTATCAGTGATAGAGAAGG
TACGTCTAGAAGGTACCCCCTATATAAGCAGAGAGATCTGTTCAA
ATTTGAACTGACTAAGCGGCTCCCGCCAGATTTTGGCAAGATTAC
TAAGCAGGAAGTCAAGGACTTTTTTGCTTGGGCAAAGGTCAATCA
GGTGCCGGTGACTCACGAGTTTAAAGTTCCCAGGGAATTGGCGGG
AACTAAAGGGGCGGAGAAATCTCTAAAACGCCCACTGGGTGACGT
CACCAATACTAGCTATAAAAGTCTGGAGAAGCGGGCCAGGCTCTC
ATTTGTTCCCGAGACGCCTCGCAGTTCAGACGTGACTGTTGATCC
CGCTCCTCTGCGACCGCTAGCTTCGATCAACTACGCAGACAGGTA
CCAAAACAAGTGTTCTCGTCACGTGGGCATTAATCTGATTCTGTT
TCCCTGCAGACAATGCGAGAGAATGAATCAGAACTCAAATATCTG
CTTCACTCACGGACAGAAAGACTGTTTAGAGTGCTTTCCCGTGTC
AGAATCTCAACCCGTTTCTGTCGTCAAAAAGGCGTATCAGAAACT
GTGCTACATTCATCATATCATGGGAAAGGTGCCAGACGCTTGCAC
TGCCTGCGATCTGGTCAATGTGGATTTGGATGACTGCATCTTTGA
ACAATAAATGACTTAAGCCAGGTATGGCTGCCGATGGTTATCTTC
CAGATTGGCTCGAGGACAACCTTAGTGAAGGAATTCGCGAGTGGT
GGGCTTTGAAACCTGGAGCCCCTCAACCCAAGGCAAATCAACAAC
ATCAAGACAACGCTAGAGGTCTTGTGCTTCCGGGTTACAAATACC
TTGGACCCGGCAACGGACTCGACAAGGGGGAGCCGGTCAACGCAG
CAGACGCGGCGGCCCTCGAGCACGACAAAGCCTACGACCAGCAGC
TCAAGGCCGGAGACAACCCGTACCTCAAGTACAACCACGCCGACG
CCGAGTTCCAGGAGCGGCTCAAAGAAGATACGTCTTTTGGGGGCA
ACCTCGGGCGAGCAGTCTTCCAGGCCAAAAAGAGGCTTCTTGAAC
CTCTTGGTCTGGTTGAGGAAGCGGCTAAGACGGCTCCTGGAAAGA
AGAGGCCTGTAGAGCAGTCTCCTCAGGAACCGGACTCCTCCGCGG
GTATTGGCAAATCGGGTGCACAGCCCGCTAAAAAGAGACTCAATT
TCGGTCAGACTGGCGACACAGAGTCAGTCCCAGACCCTCAACCAA
TCGGAGAACCTCCCGCAGCCCCCTCAGGTGTGGGATCTCTTACAA
TGGCTTCAGGTGGTGGCGCACCAGTGGCAGACAATAACGAAGGTG
CCGATGGAGTGGGTAGTTCCTCGGGAAATTGGCATTGCGATTCCC
AATGGCTGGGGGACAGAGTCATCACCACCAGCACCCGAACCTGGG
CCCTGCCCACCTACAACAATCACCTCTACAAGCAAATCTCCAACA
GCACATCTGGAGGATCTTCAAATGACAACGCCTACTTCGGCTACA
GCACCCCCTGGGGGTATTTTGACTTCAACAGATTCCACTGCCACT
TCTCACCACGTGACTGGCAGCGACTCATCAACAACAACTGGGGAT
TCCGGCCTAAGCGACTCAACTTCAAGCTCTTTAACATTCAGGTCA
AAGAGGTTACGGACAACAATGGAGTCAAGACCATCGCCAATAACC
TTACCAGCACGGTCCAGGTCTTCACGGACTCAGACTATCAGCTCC
CGTACGTGCTCGGGTCGGCTCACGAGGGCTGCCTCCCGCCGTTCC
CAGCGGACGTTTTCATGATTCCTCAGTACGGGTATCTGACGCTTA
ATGATGGAAGCCAGGCCGTGGGTCGTTCGTCCTTTTACTGCCTGG
AATATTTCCCGTCGCAAATGCTAAGAACGGGTAACAACTTCCAGT
TCAGCTACGAGTTTGAGAACGTACCTTTCCATAGCAGCTACGCTC
ACAGCCAAAGCCTGGACCGACTAATGAATCCACTCATCGACCAAT
ACTTGTACTATCTCTCTAGAACTATTAACGGTTCTGGACAGAATC
AACAAACGCTAAAATTCAGTGTGGCCGGACCCAGCAACATGGCTG
TCCAGGGAAGAAACTACATACCTGGACCCAGCTACCGACAACAAC
GTGTCTCAACCACTGTGACTCAAAACAACAACAGCGAATTTGCTT
GGCCTGGAGCTTCTTCTTGGGCTCTCAATGGACGTAATAGCTTGA
TGAATCCTGGACCTGCTATGGCCTCTCACAAAGAAGGAGAGGACC
GTTTCTTTCCTTTGTCTGGATCTTTAATTTTTGGCAAACAAGGTA
CTGGCAGAGACAACGTGGATGCGGACAAAGTCATGATAACCAACG
AAGAAGAAATTAAAACTACTAACCCGGTAGCAACGGAGTCCTATG
GACAAGTGGCCACAAACCACCAGAGTGCCCAAGCACAGGCGCAGA
CCGGTTGGGTTCAAAACCAAGGAATACTTCCGGGTATGGTTTGGC
AGGACAGAGATGTGTACCTGCAAGGACCCATTTGGGCCAAAATTC
CTCACACGGACGGCAACTTTCACCCTTCTCCGCTGATGGGAGGGT
TTGGAATGAAGCACCCGCCTCCTCAGATCCTCATCAAAAACACAC
CTGTACCTGCGGATCCTCCAACGGCCTTCAACAAGGACAAGCTGA
ACTCTTTCATCACCCAGTATTCTACTGGTCAAGTCAGCGTGGAGA
TCGAGTGGGAGCTGCAGAAGGAAAACAGCAAGCGCTGGAACCCGG
AGATCCAGTACACTTCCAACTATTACAAGTCTAATAATGTTGAAT
TTGCTGTTAATACTGAAGGTGTATATAGTGAACCCCGCCCCATTG
GCACCAGATACCTGACTCGTAATCTGTAAGTCGACTTGCTTGTTA
ATCAATAAACCGTTTAATTCGTTTCAGTTGAACTTTGGTCTCTGC
GAAGGGCAATTCGTTTAAACCTGCAGGACTAGAGGTCCTGTATTA
GAGGTCACGTGAGTGTTTTGCGACATTTTGCGACACCATGTGGTC
ACGCTGGGTATTTAAGCCCGAGTGAGCACGCAGGGTCTCCATTTT
GAAGCGGGAGGTTTGAACGCGCAGCCGCC
AAV9-iCAP AAV2 REP 1-
SEQ ID NO: 3
ATGCCGGGGTTTTACGAGATTGTGATTAAGGTCCCCAGCGACCTT
GACGAGCATCTGCCCGGCATTTCTGACAGCTTTGTGAACTGGGTG
GCCGAGAAGGAATGGGAGTTGCCGCCAGATTCTGACATGGATCTG
AATCTGATTGAGCAGGCACCCCTGACCGTGGCCGAGAAGCTGCAG
CGCGACTTTCTGACGGAATGGCGCCGTGTGAGTAAGGCCCCGGAG
GCTCTTTTCTTTGTGCAATTTGAGAAGGGAGAGAGCTACTTCCAC
ATGCACGTGCTCGTGGAAACCACCGGGGTGAAATCCATGGTTTTG
GGACGTTTCCTGAGTCAGATTCGCGAAAAACTGATTCAGAGAATT
TACCGCGGGATCGAGCCGACTTTGCCAAACTGGTTCGCGGTCACA
AAGACCAGAAATGGCGCCGGAGGCGGGAACAAGGTGGTGGATGAG
TGCTACATCCCCAATTACTTGCTCCCCAAAACCCAGCCTGAGCTC
CAGTGGGCGTGGACTAATATGGAACAGTATTTAAGCGCCTGTTTG
AATCTCACGGAGCGTAAACGGTTGGTGGCGCAGCATCTGACGCAC
GTGTCGCAGACGCAGGAGCAGAACAAAGAGAATCAGAATCCCAAT
TCTGATGCGCCGGTGATCAGATCAAAAACTTCAGCCAGGTACATG
GAGCTGGTCGGGTGGCTCGTGGACAAGGGGATTACCTCGGAGAAG
CAGTGGATCCAGGAGGACCAGGCCTCATACATCTCCTTCAATGCG
GCCTCCAACTCGCGGTCCCAAATCAAGGCTGCCTTGGACAATGCG
GGAAAGATTATGAGCCTGACTAAAACCGCCCCCGACTACCTGGTG
GGCCAGCAGCCCGTGGAGGACATTTCCAGCAATCGGATTTATAAA
ATTTTGGAACTAAACGGGTACGATCCCCAATATGCGGCTTCCGTC
TTTCTGGGATGGGCCACGAAAAAGTTCGGCAAGAGGAACACCATC
TGGCTGTTTGGGCCTGCAACTACCGGGAAGACCAACATCGCGGAG
GCCATAGCCCACACTGTGCCCTTCTACGGGTGCGTAAACTGGACC
AATGAGAACTTTCCCTTCAACGACTGTGTgGACAAGATGGTGATC
TGGTGGGAGGAGGGGAAGATGACCGCCAAGGTCGTGGAGTCGGCC
AAAGCCATTCTCGGAGGAAGCAAGGTGCGCGTGGACCAGAAATGC
AAGTCCTCGGCCCAGATAGACCCGACTCCCGTGATCGTCACCTCC
AACACCAATATGTGCGCCGTGATTGACGGGAACTCAACGACCTTC
GAACACCAGCAGCCGTTGCAAGACCGGATGTTCAAATTTGAACTC
ACCCGCCGTCTGGATCATGACTTTGGGAAGGTCACCAAGCAGGAA
GTCAAAGACTTTTTCCGGTGGGCAAAGGATCACGTGGTTGAGGTG
GAGCATGAATTCTACGTCAAAAAGGGTGGAGCCAAGAAAAGACCC
GCCCCCAGTGACGCAGATATAAGTGAGCCCAAACGGGTGCGCGAG
TCAGTTGCGCAGCCATCGACGTCAGACGCGGA
AAV9-iCAP AAV2 REP 2-
SEQ ID NO: 4
AGCTTCGATCAACTACGCGGACAGGTACCAAAACAAATGTTCTCG
TCACGTGGGCATGAATCTGATGCTGTTTCCCTGCAGACAATGCGA
GAGACTGAATCAGAATTCAAATATCTGCTTCACTCACGGTGTCAA
AGACTGTTTAGAGTGCTTTCCCGTGTCAGAATCTCAACCCGTTTC
TGTCGTCAAAAAGGCGTATCAGAAACTGTGCTACATTCATCACAT
CATGGGAAAGGTGCCAGACGCTTGCACTGCTTGCGACCTGGTCAA
TGTGGACTTGGATGACTGTGTTTCTGAACAA
AAV9-iCAP tTA-
SEQ ID NO: 5
ATGAGTCGGCTGGATAAATCTAAAGTCATAAACggcGCTCTGGAA
TTACTCAATgaaGTCGGTATCGAAGGCCTGACGACAAGGAAACTC
GCTCAAAAGCTGGGAGTTGAGCAGCCTACCCTGTACTGGCACGTG
AAGAACAAGCGGGCCCTGCTCGATGCCCTGgccATCGAGATGCTG
GACAGGCATCATACCCACTTCTGCCCCCTGGAAGGCGAGTCATGG
CAAGACTTTCTGCGGAACAACGCCAAGTCATTCCGCTGTGCTCTC
CTCTCACATCGCGACGGGGCTAAAGTGCATCTCGGCACCCGCCCA
ACAGAGAAACAGTACGAAACCCTGGAAAATCAGCTCGCGTTCCTG
TGTCAGCAAGGCTTCTCCCTGGAGAACGCACTGTACGCTCTGTCC
GCCGTGGGCCACTTTACACTGGGCTGCGTATTGGAGGAACAGGAG
CATCAAGTAGCAAAAGAGGAAAGAGAGACACCTACCACCGATTCT
ATGCCCCCACTTCTGAGACAAGCAATTGAGCTGTTCGACCGGCAG
GGAGCCGAACCTGCCTTCCTTTTCGGCCTGGAACTAATCATATGT
GGCCTGGAGAAACAGCTAAAGTGCGAAAGCGGCGGGCCGGCCGAC
GCCCTTGACGATTTTGACTTAGACATGCTCCCAGCCGATGCCCTT
GACGACTTTGACCTTGATATGCTGCCTGCTGACGCTCTTGACGAT
TTTGACCTTGACATGCTCCCCGGGTAA
AAV9-iCAP hABGH-
SEQ ID NO: 6
TATTCTATAGTGTCACCTAAATGCTAGAGCTCGCTGATCAGCCTC
GACTGTGCCTTCTAGTTGCCAGCCATCTGTTGTTTGCCCCTCCCC
CGTGCCTTCCTTGACCCTGGAAGGTGCCACTCCCACTGTCCTTTC
CTAATAAAATGAGGAAATTGCATCGCATTGTCTGAGTAGGTGTCA
TTCTATTCTGGGGGGGGGGTGGGGCAGGACAGCAAGGGGGAGGAT
TGGGAAGACAATAGCAGGCATGCTGGGGATGCGGTGGGCTCTATG
GCTTCTGAGGCGGAAAGAACCAGCTGGGGCTCGAATCAAGCT
AAV9-iCAP tet8 TRE tet binding-
SEQ ID NO: 7
TCTCCCTATCAGTGATAGAGA
AAV9-iCAP tet8 TRE AAV5 REP p41 promoter-
SEQ ID NO: 8
TGTTCAAATTTGAACTGACTAAGCGGCTCCCGCCAGATTTTGGCA
AGATTACTAAGCAGGAAGTCAAGGACTTTTTTGCTTGGGCAAAGG
TCAATCAGGTGCCGGTGACTCACGAGTTTAAAGTTCCCAGGGAAT
TGGCGGGAACTAAAGGGGCGGAGAAATCTCTAAAACGCCCACTGG
GTGACGTCACCAATACTAGCTATAAAAGTCTGGAGAAGCGGGCCA
GGCTCTCATTTGTTCCCGAGACGCCTCGCAGTTCAGACGTGACTG
TTGATCCCGCTCCTCTGCGACCGCT
AAV9-iCAP AAV2 REP 3-
SEQ ID NO: 9
AGCTTCGATCAACTACGCAGACAGGTACCAAAACAAGTGTTCTCG
TCACGTGGGCATTAATCTGATTCTGTTTCCCTGCAGACAATGCGA
GAGAATGAATCAGAACTCAAATATCTGCTTCACTCACGGACAGAA
AGACTGTTTAGAGTGCTTTCCCGTGTCAGAATCTCAACCCGTTTC
TGTCGTCAAAAAGGCGTATCAGAAACTGTGCTACATTCATCATAT
CATGGGAAAGGTGCCAGACGCTTGCACTGCCTGCGATCTGGTCAA
TGTGGATTTGGATGACTGCATCTTTGAACAATAAATGACTTAAGC
CAGGT
AAV9-iCAP tet8 TRE CAP-
SEQ ID NO: 10
ATGGCTGCCGATGGTTATCTTCCAGATTGGCTCGAGGACAACCTT
AGTGAAGGAATTCGCGAGTGGTGGGCTTTGAAACCTGGAGCCCCT
CAACCCAAGGCAAATCAACAACATCAAGACAACGCTaGAGGTCTT
GTGCTTCCGGGTTACAAATACCTTGGACCCGGCAACGGACTCGAC
AAGGGGGAGCCGGTCAACGCAGCAGACGCGGCGGCCCTCGAGCAC
GACAAaGCCTACGACCAGCAGCTCAAGGCCGGAGACAACCCGTAC
CTCAAGTACAACCACGCCGACGCCGAGTTCCAGGAGCGGCTCAAA
GAAGATACGTCTTTTGGGGGCAACCTCGGGCGAGCAGTCTTCCAG
GCCAAAAAGAGGCTTCTTGAACCTCTTGGTCTGGTTGAGGAAGCG
GCTAAGACGGCTCCTGGAAAGAAGAGGCCTGTAGAGCAGTCTCCT
CAGGAACCGGACTCCTCCGCGGGTATTGGCAAATCGGGTGCACAG
CCCGCTAAAAAGAGACTCAATTTCGGTCAGACTGGCGACACAGAG
TCAGTCCCAGACCCTCAACCAATCGGAGAACCTCCCGCAGCCCCC
TCAGGTGTGGGATCTCTTACAATGGCTTCAGGTGGTGGCGCACCA
GTGGCAGACAATAACGAAGGTGCCGATGGAGTGGGTAGTTCCTCG
GGAAATTGGCATTGCGATTCCCAATGGCTGGGGGACAGAGTCATC
ACCACCAGCACCCGAACCTGGGCCCTGCCCACCTACAACAATCAC
CTCTACAAGCAAATCTCCAACAGCACATCTGGAGGATCTTCAAAT
GACAACGCCTACTTCGGCTACAGCACCCCCTGGGGGTATTTTGAC
TTCAACAGATTCCACTGCCACTTCTCACCACGTGACTGGCAGCGA
CTCATCAACAACAACTGGGGATTCCGGCCTAAGCGACTCAACTTC
AAGCTCTTLAACATTCAGGTCAAAGAGGTTACGGACAACAATGGA
GTCAAGACCATCGCCAATAACCTTACCAGCACGGTCCAGGTCTTC
ACGGACTCAGACTATCAGCTCCCGTACGTGCTCGGGTCGGCTCAC
GAGGGCTGCCTCCCGCCGTTCCCAGCGGACGTTTTCATGATTCCT
CAGTACGGGTATCTGACGCTTAATGATGGAAGCCAGGCCGTGGGT
CGTTCGTCCTTTTACTGCCTGGAATATTTCCCGTCGCAAATGCTA
AGAACGGGTAACAACTTCCAGTTCAGCTACGAGTTTGAGAACGTA
CCTTTCCATAGCAGCTACGCTCACAGCCAAAGCCTGGACCGACTA
ATGAATCCACTCATCGACCAATACTTGTACTATCTCTCTAGAACT
ATTaACGGttCtGgacagaatcaaCAAACGCTAAAATTCAGTGTG
GCCGGACCCAGCAACATGGCTGTCCAGGGAAGAAACTACATACCT
GGACCCAGCTACCGACAACAACGTGTCTCAACCACTGTGACTCAA
AACAACAACAGCGAATTTGCTTGGCCTGGAGCTTCTTCTTGGGCT
CTCAATGGACGTAATAGCTTGATGAATCCTGGACCTGCTATGGCC
tctCACAAAGAAGGAGAGGACCGTTTCTTTCCTTTGTCTGGATCT
TTAATTTTTGGCAAACAAGGTACtGGCAGAGACAACGTGGATGCG
GACAAAGTCATGATAACCAACGAAGAAGAAATTAAAACTACTAAC
CCGGTAGCAACGGAGTCCTATGGACAAGTGGCCACAAACCACCAG
AGTGCcCAAGCACAGGCGCAGACCGGTTGGGTTCAAAACCAAGGA
ATACTTCCGGGTATGGTTTGGCAGGACAGAGATGTGTACCTGCAA
GGACCCATTTGGGCCAAAATTCCTCACACGGACGGCAACTTTCAC
CCTTCTCCGCTGATGGGAGGGTTTGGAATGAAGCACCCGCCTCCT
CAGATCCTCATCAAAAACACACCTGTACCTGCGGATCCTCCAACG
GCCTTCAACAAGGACAAGCTGAACTCTTTCATCACCCAGTATTCT
ACTGGICAAGTCAGCGTGGAGATCGAGTGGGAGCTGCAGAAGGAA
AACAGCAAGCGCTGGAACCCGGAGATCCAGTACACTTCCAACTAT
TACAAGTCTAATAATGTTGAATTTGCTGTTAATACTGAAGGTGTA
TATAGTGAACCCCGCCCCATTGGCACCAGATACCTGACTCGTAAT
CTG
AAV9-iCAP AAV 2 and p5-
SEQ ID NO: 11
taagtegacTTGCTTGTTAATCAATAAACCGTTTAATTCGTTTCA
GTTGAACTTTGGTCTCTGCGAAGGGCAATTCgTTTAAACCTGCAG
GACTAGAGGTCCTGTATTAGAGGTCACGTGAGTGTTTTGCGACAT
TTTGCGACACCATGTGGTCACGCTGGGTATTTAAGCCCGAGTGAG
CACGCAGGGTCTCCATTTTGAAGCGGGAGGTTTGAACGCGCAGCC
GCC
AAV9-iCAP tet6 TRE tet binding-
SEQ ID NO: 12
TCCCTATCAGTGATAGAGA

### EXAMPLES

### Example 1—Adherent Cell Vector Production Process for Production of Capsid Variants

HEK293 cells are seeded prior to transfection. The plasmids and transfection reagent are diluted, vortexed and incubated before being added to the cells. At the determined timepoint post-transfection, cells are dislodged in media then separated by centrifugation. Cells are lysed to release AAV particles with lysis buffer containing salt, magnesium chloride, detergent and nuclease. The lysate is then clarified by centrifugation. Virus secreted in the media is precipitated using polyethylene glycol (PEG), digested with nuclease, and combined with the clarified cell lysate. Clarified and nuclease-treated crude lysates are overlaid on a discontinuous iodixanol gradient and separated by high-speed ultra-centrifugation. Full vectors are then removed by inserting a needle through the tubing and extracting the virus present in the 40%/60% iodixanol axis. These vectors are then concentrated, and buffer exchanged using an centrifugal filter.

### Example 2—Suspension and Manufacturing Process

Suspension HEK293 cells are scaled up and transfected when they reach the target density. The plasmids and transfection reagent are diluted, vortexed and incubated before being added to the cells. At the determined timepoint post-transfection, cells are lysed with detergent and nuclease and clarified by depth filtration. Clarified material is purified by affinity chromatography. Neutralized elution product is concentrated by tangential flow filtration and enriched for full capsids by cesium ultra-centrifugation. Enriched capsids are concentrated, and buffer exchanged by ultracentrifugation and diafiltration to the final target concentration.

### Example 3—Production of Tet8 Derived TRE Plasmid rAAVs

Using the methods outlined in Examples 1 and 2, rAAVs were produced in HEK293 cells using plasmids comprising a tetracycline-controlled transactivator (tTA)-Tetracycline response element (TRE)-based inducible amplification loop to increase virus production. In these exemplified rAAVs, the plasmid TRE was a tet8 derived TRE. Table 1, below, provides the rAAVs produced using the methods of Examples 1 and 2. As shown in Table 1, many of these rAAVs included genes included by the plasmids.

TABLE 1

| Capsid variant | Included gene(s) in plasmid |
|---|---|
| AAV9 | Enhanced green fluorescent protein (eGFP), |
| | human iduronate 2 sulfatase (hIDS), syntaxin- |
| | binding protein 1 (STXBP1), Gaussia-DuraLuc |
| | (gDura-Luc) |
| AAV9-B10 | eGFP |
| AAV9-B22 | eGFP |
| AAV9-C1 | eGFP, hIDS |
| AAV9-C4 | eGFP, hIDS |
| AAV9-C5 | eGFP, hIDS, gDura-Luc |
| AAV9-D2 | eGFP |
| AAV9-D3 | eGFP |
| AAV9-D6 | eGFP, hIDS |
| AAV9-D7 | eGFP |
| AAV9-D8 | eGFP |
| AAV9-D9 | eGFP |
| AAV9-D10 | eGFP |
| AAV9-D11 | eGFP |
| AAV9-E1.13 | eGFP, STXBP1, Frataxin (FXN) |
| AAV9-E4.1 | FXN |
| AAV9-E6 | eGFP |
| AAV9-E7 | eGFP |

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

What is claimed is:

1. An adeno-associated virus (AAV) rep-cap plasmid comprising:
a rep gene or portions thereof;
a cap gene;
a tetracycline-inducible expression system in between the rep gene and the cap gene; and
an AAV p41 promoter,
wherein the AAV rep-cap plasmid comprising a nucleic acid sequence selected from the group consisting of SEQ ID NO: 1 and SEQ ID NO: 2.

2. The plasmid of claim 1 comprising SEQ ID NO: 1.

3. The plasmid of claim 1 comprising SEQ ID NO: 2.

4. The plasmid of claim 1 further comprising one or more helper genes.

5. The plasmid of claim 4 wherein the one or more helper genes are selected from the group consisting of E4, E2a and VA.

6. A method of manufacturing an adeno-associated virus (AAV), the method comprising:
introducing an AAV rep-cap plasmid into a cell.

7. The method of claim 6 wherein the AAV rep-cap plasmid comprises SEQ ID NO: 1.

8. The method of claim 6 wherein the AAV rep-cap plasmid comprises SEQ ID NO: 2.

9. The method of claim 6 further comprising introducing a helper plasmid comprising one or more helper genes into the cell.

10. The method of claim 9 wherein the one or more helper genes are selected from the group consisting of E4, E2a and VA.

11. The method of claim 6 wherein the AAV rep-cap plasmid further comprises one or more helper genes.

12. The method of claim 11 wherein the one or more helper genes are selected from the group consisting of E4, E2a and VA.

13. The method of claim 6 further comprising introducing a plasmid comprising a gene of interest flanked by inverse terminal repeats.

14. The method of claim 13 wherein the gene of interest is a transgene.

15. The method of claim 13 wherein the gene of interest encodes a protein associated with a disease.

16. The method of claim 6 wherein the cell is mammalian.

17. The method of claim 16 wherein the cell is immortalized.

18. The method of claim 17 wherein the immortalized cell is an embryonic stem cell.

19. The method of claim 18 wherein the embryonic stem cell is a human embryonic stem cell.

20. The method of claim 19 wherein the human embryonic stem cell is a human embryonic kidney 293 (HEK-293) cell.


SEQUENCE LISTING (12 sequences)

SEQ ID NO: 1 - DNA, 6345 bp, Artificial Sequence
  Synthesized
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggtatggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 2 - DNA, 6284 bp, Artificial Sequence
  Synthesized
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggtatgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaaatgcatgaattcgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagctatcaagtgccacctgacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaacgtctccctatcagtgatagagaagtcgacacgtctcgagctccctatcagtgatagagaaggtacgtctagaaggtaccccctatataagcagagagatctgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctagcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggtatggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 3 - DNA, 1562 bp, Artificial Sequence
  Synthesized
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaatatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcgga

SEQ ID NO: 4 - DNA, 301 bp, Artificial Sequence
  Synthesized
agcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaa

SEQ ID NO: 5 - DNA, 747 bp, Artificial Sequence
  Synthesized
atgagtcggctggataaatctaaagtcataaacggcgctctggaattactcaatgaagtcggtatcgaaggcctgacgacaaggaaactcgctcaaaagctgggagttgagcagcctaccctgtactggcacgtgaagaacaagcgggccctgctcgatgccctggccatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 6 - DNA, 313 bp, Artificial Sequence
  Synthesized
tattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaatcaagct

SEQ ID NO: 7 - DNA, 21 bp, Artificial Sequence
  Synthesized
tctccctatcagtgatagaga

SEQ ID NO: 8 - DNA, 295 bp, Artificial Sequence
  Synthesized
tgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgct

SEQ ID NO: 9 - DNA, 320 bp, Artificial Sequence
  Synethized
agcttcgatcaactacgcagacaggtaccaaaacaagtgttctcgtcacgtgggcattaatctgattctgtttccctgcagacaatgcgagagaatgaatcagaactcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaagccaggt

SEQ ID NO: 10 - DNA, 2208 bp, Artificial Sequence
  Synthesized
atggctgccgatggttatcttccagattggctcgaggacaaccttagtgaaggaattcgcgagtggtgggctttgaaacctggagcccctcaacccaaggcaaatcaacaacatcaagacaacgctagaggtcttgtgcttccgggttacaaataccttggacccggcaacggactcgacaagggggagccggtcaacgcagcagacgcggcggccctcgagcacgacaaagcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctctttaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggcctctcacaaagaaggagaggaccgtttctttcctttgtctggatctttaatttttggcaaacaaggtactggcagagacaacgtggatgcggacaaagtcatgataaccaacgaagaagaaattaaaactactaacccggtagcaacggagtcctatggacaagtggccacaaaccaccagagtgcccaagcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggtcaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 11 - DNA, 228 bp, Artificial Sequence
  Synthesized
taagtcgacttgcttgttaatcaataaaccgtttaattcgtttcagttgaactttggtctctgcgaagggcaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgcc

SEQ ID NO: 12 - DNA, 19 bp, Artificial Sequence
  Synthesized
tccctatcagtgatagaga